(19) United States
(12) Patent Application Publication
(10) Pub. No.: US 2022/0155196 A1
(43) Pub. Date: May 19, 2022

(54) IN-SITU SURROUNDING ROCK TESTING DEVICE AND METHOD

(71) Applicant: Tongji University, Shanghai (CN)

(72) Inventors: Yadong Xue, Shanghai (CN); Rundong Zhang, Shanghai (CN); Jiaxuan Wang, Shanghai (CN); Yongqiang Fan, Shanghai (CN); Hongfei Zhang, Shanghai (CN); Mingliang Zhou, Shanghai (CN); Liyun XUE, Shanghai (CN)

(21) Appl. No.: 17/375,157

(22) Filed: Jul. 14, 2021

(30) Foreign Application Priority Data
Nov. 18, 2020 (CN) 202011293972.6

(51) Int. Cl. G01N 3/06; G01N 3/08

(52) U.S. Cl. CPC G01N 3/06; G01N 3/08; G01N 2203/0075; G01N 2203/0019; G01N 2203/0003

(57) ABSTRACT

This disclosure describes an in-situ surrounding rock testing device and method. The testing device includes a collection device and a control terminal. The collection device includes a pressure cell, displacement meters and a magnetic base. When mechanical properties of surrounding rock are tested, the collection device is only necessary to be installed on an outer surface of a gripper of a TBM. The outer surface of the gripper is coupled to a rear end surface of the magnetic base; a front end surface of the pressure cell and displacement meters are in contact with the surrounding rock. The pressure cell measures pressures undergone by the surrounding rock. The displacement meters measure a total compaction displacement of the surrounding rock relative to the collection device. A pressure-displacement curve of the surrounding rock can be obtained by the testing device while pressing the gripper tightly against the surrounding rock.

### CROSS REFERENCE TO RELATED APPLICATION

This patent application claims the benefit and priority of Chinese Patent Application No. 202011293972.6, titled “In-Situ Surrounding Rock Testing Device and Method”, filed on Nov. 18, 2020, the disclosure of which is incorporated by reference herein in its entirety.

### TECHNICAL FIELD

The present disclosure relates to a field of surrounding rock testing, in particular to an in-situ surrounding rock testing device and method.

### BACKGROUND ART

With rapid development of social economy and construction technology, and in view of urgent needs for development of transportation and water conservancy, there are a large number of hydraulic tunnels, railway tunnels, traffic tunnels and various pipeline tunnels constructed through a TBM (Tunnel Boring Machine) method. However, the TBM is extremely sensitive to conditions of the surrounding rock, and strength of the surrounding rock directly affects boring efficiency and wear degree of a cutter disk of the TBM. During a process of TBM tunnel construction, because of discontinuity of geological survey for the rock strength along a tunnel, and the lag of laboratory test results of the rock strength, there are low tunnel construction efficiency and high construction cost, and even other construction problems may occur, such as the TBM jamming. Therefore, obtaining real-time, continuous and accurate strength parameters of the surrounding rock along a tunnel during TBM tunneling process and timely providing a feedback to constructors are significant for ensuring the working efficiency of TBM and the safety of constructors and machines.

At present, traditional methods for obtaining strength parameters of the in-situ surrounding rock in a TBM tunnel mainly need to drill the surrounding rock to obtain a core on site. The core is manufactured as a standard rock sample by cutting and polishing, and finally an indoor test is performed on the sample to obtain mechanical properties of the rock. In recent years, some devices and methods have developed which are used to perform the in-situ test at the construction site.

Chinese patent application No. 201210309656.2 provides an integrated collection device to obtain a stress and a displacement of a surrounding rock. Multiple spheres with an elastic modulus close to that of the surrounding rock to be tested are embedded in a borehole, and gaps are filled with grouting. By doing this, strains in six directions at a certain point are measured so as to calculate a three-dimensional stress at this point, thereby obtaining a stress and a displacement of the tested point.

Chinese patent application No. 201610823130.4 provides an on-line identification method for surrounding rock strength used in a TBM. Through sensing information from the TBM, in combination with an existing surrounding rock cutting model, strength of the surrounding rock on an excavation face is identified.

Chinese patent application No. 201811177140.0 provides a quick in-situ testing method for strength of a surrounding rock in a tunnel constructed by a tunnel boring machine. By passing a rock tester through a manhole in the cutter head of the tunnel boring machine, an in-situ strength test is performed on the surrounding rock to obtain data through a “four-line and four-point” method, and the surrounding rock strength is obtained by calculation.

Chinese patent application No. 201910979186.2 provides an automatic testing system and method for mechanical parameters of a surrounding rock which is suitable for a TBM. A robot grabs excavated rock debris conveyed on a belt conveyor for an on-site abrasion test and a compressive strength test, thereby obtaining mechanical parameters of the surrounding rock including strength of the surrounding rock.

There is a serious lag when mechanical properties of the surrounding rock are obtained by a traditional core-drilling laboratory testing method, and a test process takes a long time; when a core is drilled near a tunnel face, a normal operation of a TBM is affected, thus the traditional laboratory testing method cannot meet requirements for rapid and real-time testing on mechanical properties of the rock during construction of the TBM. The on-line identification method of the surrounding rock strength uses a surrounding rock cutting model to calculate the surrounding rock strength, but the accuracy and precision of the results need to be further verified. With respect to the in-situ rapid testing method by passing a rock tester through a manhole in the cutter head of a tunnel boring machine, the cutter head needs to be rotated so that the manhole of the cutter head is stopped sequentially at a position of the tunnel face that is in directions of 3, 6, 9, 12 o'clock, which seriously affects a normal working of a TBM and greatly reduces tunneling efficiency. With respect to an automatic testing system in which a robot grabs rock debris on a belt conveyor. Because mechanical properties of the cut and extruded rock debris may change, there is a big error between test results of the rock debris and actual strength of the surrounding rock. In general, the existing testing devices or methods cannot simultaneously have in-situ, rapid, real-time and accurate characteristics, as well as no impact on construction when testing mechanical properties of the surrounding rock.

### SUMMARY

A purpose of the present disclosure is to provide an in-situ surrounding rock testing device and method, which can perform an in-situ testing on surrounding rock in real time, rapidly and accurately.

In order to achieve the above purpose, the present disclosure provides the following solutions.

An in-situ surrounding rock testing device is disclosed, said testing device includes a collection device and a control terminal.

The collection device includes a pressure cell, multiple displacement meters and a magnetic base.

A rear end surface of the pressure cell and the multiple displacement meters are fixed on a front end surface of the magnetic base.

When the testing device tests a surrounding rock, a rear end surface of the magnetic base is attracted to an outer surface of a gripper of a tunnel boring machine; a front end surface of the pressure cell and the multiple displacement meters are in contact with the surrounding rock; the pressure cell is configured to measure pressures to which the surrounding rock is subjected; and the multiple displacement meters are configured to measure total compaction displacements of the surrounding rock relative to the collection device.

The pressure cell and the multiple displacement meters are connected to the control terminal; the control terminal is configured to: synchronously collect the pressures measured by the pressure cell and the total compaction displacements measured by the multiple displacement meters; based on the pressures and the total compaction displacements, determine a pressure-displacement curve of the surrounding rock and a slope of a point on the pressure-displacement curve corresponding to a maximum pressure; and obtain a compressive strength of the surrounding rock based on the slope.

Optionally, the collection device further includes a load-bearing plate.

A bottom surface of the load-bearing plate is fixedly connected with the front end surface of the pressure cell.

When the testing device tests the surrounding rock, a top surface of the load-bearing plate is in contact with the surrounding rock.

Optionally, the collection device further includes a mounting rod and a mounting rod support.

The mounting rod support is arranged on the magnetic base.

The mounting rod is connected with the mounting rod support by a bolt, and the collection device is fixedly installed on the outer surface of the gripper through the mounting rod.

Optionally, the mounting rod includes a telescopic device rod, a telescopic hand-held rod, an end connector, a rotating bearing, a corner connector, and a mounting rod handle.

The rotating bearing is arranged on the end connector; the rotating bearing enables the end connector to rotate relative to the telescopic device rod.

An end of the telescopic device rod is connected with an end of the telescopic hand-held rod through the corner connector; another end of the telescopic device rod is connected with the rotating bearing; and another end of the telescopic hand-held rod is connected with the mounting rod handle.

The end connector is fixedly connected with the mounting rod support.

Optionally, the control terminal includes a controller, an input device, a memory, a microprocessor, a display, and a battery box.

The controller is respectively connected with the pressure cell, the multiple displacement meters and the memory; the controller is configured to synchronously collect the pressures measured by the pressure cell and the total compaction displacements measured by the multiple displacement meters, and configured to transmit the pressures and the total compaction displacements synchronously collected to the memory for storage.

The input device is connected with the memory; the input device is configured to obtain a correspondence table among the slope, an elastic modulus and the compressive strength, and to obtain range information of the surrounding rock; and configured to store the correspondence table among the slope, the elastic modulus and the compressive strength, as well as the range information of the surrounding rock in the memory.

The microprocessor is connected to the memory; the microprocessor is configured to obtain the pressures and total compaction displacements at all collection time points from the memory, and configured to determine the pressure-displacement curve of the surrounding rock based on the pressures and the total compaction displacements at all collection time points and to determine the slope of the point on the pressure-displacement curve corresponding to a maximum pressure; the microprocessor is also configured to obtain an elastic modulus and a compressive strength corresponding to the slope by referring the correspondence table among the slope, the elastic modulus and the compressive strength, and configured to transmit the pressure-displacement curve, the slope, the elastic modulus, and the compressive strength to the memory for storage.

The microprocessor is also connected to the display; the microprocessor is further configured to transmit the pressures and the total compaction displacements at all collection time points, the pressure-displacement curve, the slope, the elastic modulus, and the compressive strength to the display for displaying.

A power input end of the controller, a power input end of the memory, a power input end of the microprocessor, and a power input end of the display are connected to an input end of an integrated power.

The battery box is respectively connected with the pressure cell, the multiple displacement meters, the magnetic base and the input end of the integrated power.

Optionally, the controller includes an integrated chip, a main switch and multiple sub-switches.

The battery box is connected to an input end of the main switch; an output end of the main switch is connected to an input end of each of the multiple sub-switches; output ends of the multiple sub-switches are connected with the pressure cell, the multiple displacement meters, the magnetic base and the input end of the integrated power in one-to-one correspondence.

A control end of the main switch and a control end of each of the multiple sub-switches are connected to the integrated chip.

One embodiment discusses a method for in-situ surrounding rock testing, the testing method includes the following steps:

a performing step, configured for performing a uni-axial compression test respectively on a pressure cell, a magnetic base, and a load-bearing plate of an in-situ surrounding rock testing device, and respectively obtaining a pressure-displacement relationship curve of the pressure cell, a pressure-displacement relationship curve of the magnetic base, and a pressure-displacement relationship curve of the load-bearing plate;

a placing step, configured for placing the in-situ surrounding rock testing device at a gripper of a tunnel boring machine, and pressing the in-situ surrounding rock testing device and the surrounding rock tightly through the gripper of the tunnel boring machine;

a first obtaining step, configured for obtaining a pressure measured by the pressure cell and a total compaction displacement measured by multiple displacement meters at each collection time point;

a taking step, configured for taking a product of the pressure measured by the pressure cell at each collection time point and a cross-sectional area of the pressure cell, as a pressure of the surrounding rock at each collection time point;

a first determining step, configured for determining a displacement of the pressure cell, a displacement of the magnetic base, and a displacement of the load-bearing plate at each collection time point based on the pressure measured by the pressure cell at each collection time point, by utilizing the pressure-displacement relationship curve of the pressure cell, the pressure-displacement relationship curve of the magnetic base and the pressure-displacement relationship curve of the load-bearing plate;

a second determining step, configured for determining a displacement of the surrounding rock at each collection time point based on the total compaction displacement measured by the multiple displacement meters at each collection time point as well as the displacement of the pressure cell, the displacement of the magnetic base and the displacement of the load-bearing plate at each collection time point;

a third determining step, configured for determining a pressure-displacement curve of the surrounding rock and a slope at a point of the pressure-displacement curve corresponding to a maximum pressure based on the pressure of the surrounding rock and the displacement of the surrounding rock;

a drilling step, configured for drilling a core at the surrounding rock tested by the in-situ surrounding rock testing device;

a second obtaining step, configured for a correspondence table among the slope, an elastic modulus and a compressive strength through an indoor test; and

a third obtaining step, configured for obtaining the elastic modulus and the compressive strength corresponding to the slope based on the slope, by means of the correspondence table among the slope, the elastic modulus and the compressive strength.

In the third determining step, determining a partial pressure-displacement curve of the surrounding rock and a slope at a point of the partial pressure-displacement curve corresponding to a maximum pressure based on the pressure of the surrounding rock and the displacement of the surrounding rock.

In the drilling step, drilling a core at the surrounding rock tested by the in-situ surrounding rock testing device, to obtain an elastic modulus and a compressive strength of the surrounding rock by performing an indoor laboratory test on the core, and in turn to obtain a global pressure-displacement curve and a slope of the global pressure-displacement curve of the surrounding rock based on the partial pressure-displacement curve.

In the second obtaining step, obtaining a correspondence table among the elastic modulus, the compressive strength and the slope of the global pressure-displacement curve of the surrounding rock by repeating the drilling step.

In the third obtaining step, obtaining an elastic modulus and a compressive strength of a surrounding rock at a new location to be detected, based on the slope and the global pressure-displacement curve by means of the correspondence table. Optionally, determining a displacement of the surrounding rock at each collection time point, based on the total compaction displacement measured by the multiple displacement meters at each collection time point, as well as the displacement of the pressure cell, the displacement of the magnetic base and the displacement of the load-bearing plate at each collection time point, includes:

determining a displacement of the surrounding rock at each collection time point, based on the total compaction displacement measured by the multiple displacement meters at each collection time point, the displacement of the pressure cell, the displacement of the magnetic base and the displacement of the load-bearing plate at each collection time point, through the following equation:

$$X = \frac{{\sum }_{i = 1}^{n} {X}_{0 i}}{n} - {X}_{1} - {X}_{2} - {X}_{3} - {X}_{4} \qquad ( 1 )$$

Where X is the displacement of the surrounding rock at each collection time point; X₀ᵢ is the total compaction displacement measured by the i^{th} displacement meter at each collection time point, n is a number of displacement meters; X₁ is a displacement of the pressure cell at each collection time point; X₂ is a displacement of the magnetic base at each acquisition time point; X₃ is a displacement of the load-bearing plate at each collection time point; and X₄ is an average displacement of the multiple displacement meters when an indicating value of the pressure cell is not zero during the test.

Optionally, after the determining a displacement of the surrounding rock at each collection time point based on the total compaction displacement measured by the multiple displacement meters at each collection time point, as well as the displacement of the pressure cell, the displacement of the magnetic base and the displacement of the load-bearing plate at each collection time point, the testing method further includes:

when the displacement of the surrounding rock is equal to a maximum displacement threshold or a pressure of the surrounding rock is equal to 100 MPa, the test is stopped.

Optionally, an equation for calculating the slope of the point on the pressure-displacement curve corresponding to the maximum pressure is:

$$k = \frac{F}{X} \qquad ( 2 )$$

Where k is the slope of the point on the pressure-displacement curve corresponding to the maximum pressure; F is the maximum pressure on the pressure-displacement curve; and X is the displacement of the surrounding rock corresponding to the maximum pressure on the pressure-displacement curve.

According to the specific embodiments provided by the present disclosure, the present disclosure provides the following technical effects.

The present disclosure provides an in-situ surrounding rock testing device and method. The testing device includes a collection device and a control terminal. The collection device includes a pressure cell, multiple displacement meters and a magnetic base. The testing device has a simple structure. When mechanical properties of surrounding rock are tested, it is only necessary to install the collection device on an outer surface of a gripper of a TBM. A rear end surface of the magnetic base of the collection device is attracted on the outer surface of the gripper; a front end surface of the pressure cell and the multiple displacement meters are all in contact with the surrounding rock. The pressure cell measures a pressure undergone by the pressure cell, the displacement meters measure a total compaction displacement of the collection device relative to the surrounding rock. A pressure-displacement curve of the surrounding rock and a slope thereof can be obtained by the testing device through a process of the gripper being pressed tightly against the surrounding rock, which can help constructors intuitively evaluate strength of the surrounding rock to be tested. Furthermore, by looking up a correspondence table among the slope, an elastic modulus and a compressive strength based on the specific slope of a current rock, an elastic modulus and a compressive strength of the current rock mass can be obtained. This disclosure realizes in-situ testing for surrounding rock in a real-time and in an accurate manner without affecting normal construction and with minimal damage to surrounding rock of an excavated tunnel.

### BRIEF DESCRIPTION OF THE DRAWINGS

Various objects and advantages and a more complete understanding of the present disclosure are apparent and more readily appreciated by referring to the following detailed description and to the appended claims when taken in conjunction with the accompanying drawings:

FIG. 1 is a structural schematic diagram of a collection device provided by the present disclosure;

FIG. 2 is a diagram showing an installation location of the collection device provided by the present disclosure;

FIG. 3 is a schematic diagram of a load-bearing plate provided by the present disclosure;

FIG. 4 is a structural schematic diagram showing a retracted state of a mounting rod provided by the present disclosure;

FIG. 5 is a structural schematic diagram showing an extended state of the mounting rod provided by the present disclosure;

FIG. 6 is a structural schematic diagram showing an exploded state of the mounting rod provided by the present disclosure;

FIG. 7 is a schematic diagram of a corner connector provided by the present disclosure;

FIG. 8 is a schematic diagram of a rotating bearing provided by the present disclosure;

FIG. 9 shows a method of controlling an in-situ surrounding rock testing device according to an embodiment of the present disclosure;

FIG. 10 shows a method in-situ surrounding rock testing according to an embodiment of the present disclosure;

List of reference numbers: 1 pressure cell, 2 displacement meter, 3 magnetic base, 4 load-bearing plate, 5 mounting rod support, 6 collective wire, 7 gripper, 8 collection device, 9 mounting rod, 9-1 end connector, 9-2 rotating bearing, 9-3 telescopic device rod, 9-4 corner connector, 9-5 telescopic hand-held rod, 9-6 mounting rod handle.

### DETAILED DESCRIPTION OF THE EMBODIMENTS

The technical solutions in the embodiments of the present disclosure will be clearly and completely described below in conjunction with the accompanying drawings in the embodiments of the present disclosure. The described embodiments are only a part of the embodiments of the present disclosure, and do not represent all possible embodiments of the disclosure. Based on the embodiments of the present disclosure, all other embodiments obtained by those of ordinary skill in the art without undue experimentation fall within the scope of the present disclosure.

A purpose of the present disclosure is to provide an in-situ surrounding rock testing device and method, which can perform in-situ testing on a surrounding rock in real time and accurately.

To achieve the above purpose and to more clearly articulate the features and advantages of the present disclosure, the present disclosure will be further described in detail below with reference to the accompanying drawings and specific embodiments.

The present disclosure aims to provide an in-situ testing device that can be configured to obtain a relationship between a pressure and a displacement of surrounding rock of a tunnel in real time at a TBM working site. When a TBM is shut down, through a physical process in which a gripper of the TBM presses tightly against the surrounding rock of an excavated tunnel, a pressure-displacement curve of surrounding rock is obtained in-situ through the testing device of the present disclosure placed on a surface of the gripper of the TBM, and then mechanical properties of the surrounding rock can be obtained by analysis of the pressure-displacement curve of the surrounding rock. In this way, a reference basis is provided for a TBM operator to set reasonable tunneling parameters, which improves TBM working efficiency, reduces cost caused by frequent replacement of worn hobs, and enhances working safety of TBM.

An in-situ surrounding rock testing device provided by the present disclosure includes a collection device 8 and a control terminal.

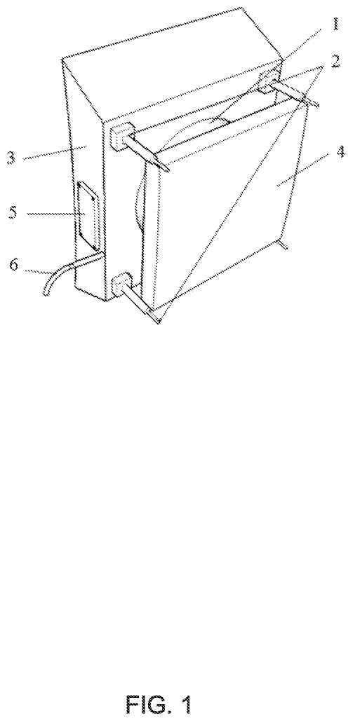

The collection device 8 includes a pressure cell 1, multiple displacement meters 2 and a magnetic base 3, as shown in FIG. 1.

A rear end surface of the pressure cell 1 and the multiple displacement meters 2 are all fixed on a front end surface of the magnetic base 3. In some embodiments, the pressure cell 1 is fixed at a center of a front surface of the magnetic base 3. The pressure cell 1 is a cylinder and can have a diameter of about 12 cm and a height of about 5-7 cm. A vibrating wire strain gauge can be used. A range of the pressure cell 1 can be not less than 110 MPa, and an accuracy of the pressure cell may not be less than 0.1 MPa. A manufactured pressure cell 1 should be first calibrated in a laboratory to determine a relationship between a bearing-pressure and a deformation of the pressure cell, so as to avoid influence on test results caused by deformation of the pressure cell 1. The collection device 8 can be provided with four displacement meters 2, which are respectively installed at four corners on the front end surface of the magnetic base 3. The displacement meters 2 are a miniature linear displacement sensor. A range of each of the displacement meters 2 should be in a range of 0-20 mm, and the accuracy thereof should reach 0.01 mm. A diameter of each of the displacement meters should be no more than 20 mm, and a height thereof should exceed a sum of a height of the pressure cell 1 and a height of the load-bearing plate 4, and the excess amount should be no more than 20 mm.

When the testing device tests the surrounding rock, an outer surface of a gripper 7 of the tunnel boring machine is attracted onto a rear end surface of the magnetic base 3. The front end surface of the pressure cell 1 and the multiple displacement meters 2 are all in contact with the surrounding rock. The pressure cell 1 is configured to measure a pressure undergone by the surrounding rock. The displacement meters 2 are configured to measure a total compaction displacement of the surrounding rock relative to the collection device 8.

The pressure cell 1 and the multiple displacement meters 2 are all connected with the control terminal. The control terminal is configured to: synchronously collect a pressure measured by the pressure cell 1 and the total compaction displacement measured by the multiple displacement meters 2; based on the pressure and the total compaction displacement, determine a pressure-displacement curve of the surrounding rock and a slope at a point corresponding to a maximum pressure on the pressure-displacement curve; and obtain a compressive strength of the surrounding rock based on the slope. In some embodiments, in a test, when a pressure undergone by the pressure cell 1 is not zero, the displacement meters 2 and the pressure cell 1 start to simultaneously collect and store test data at a fixed time interval (0.1 s). When a displacement of the surrounding rock reaches Xm or a pressure measured by the pressure cell reaches 100 MPa, the test is stopped, where Xm is a maximum displacement that does not affect a bearing capacity of the surrounding rock, which should be determined according to specific conditions on site.

The magnetic base 3 couples the collection device 8 to the outer surface of the gripper 7, and can include a DC electromagnet, a wire and a shell.

The shell of the magnetic base 3 can have a length of about 20 cm, a width of about 20 cm, and a height of about 5 cm. The shell of the magnetic base 3 can be made of SPCC cold-rolled sheet to form a closed space. A surface of the magnetic base 3 that is in contact with the gripper 7 can be a curved surface. A curvature of the curved surface is the same as that of gripper 7, which ensures that the magnetic base 3 can be closely fitted to the gripper 7 after the magnetic base 3 is energized. A surface opposite to the curved surface is a flat surface which is provided with recesses for installing the displacement meters 2 and the pressure cell 1.

An iron core of the electromagnet can be made of soft magnetic materials with high permeability such as industrial pure iron, and wound with a copper coil on an outside thereof. A wire connected externally with the copper coil is led out through a side of the shell of the magnetic base which is connected to the mounting rod 9, and connected to a power supply in the control terminal.

In order to ensure that the magnetic base 3 does not undergo significant deformation, a strength of the shell should be no less than 350 MPa. A suction force of the iron core of the electromagnet should be no less than 1.5 times a total weight of the testing device, so as to ensure that the testing device coupled to the outer surface of the gripper 7 does not slide and/or fall off. A manufactured magnetic base 3 should be calibrated in the laboratory first to determine a relationship curve between a bearing load and a deformation of the magnetic base, so as to avoid influence on test results caused by deformation of the magnetic base 3.

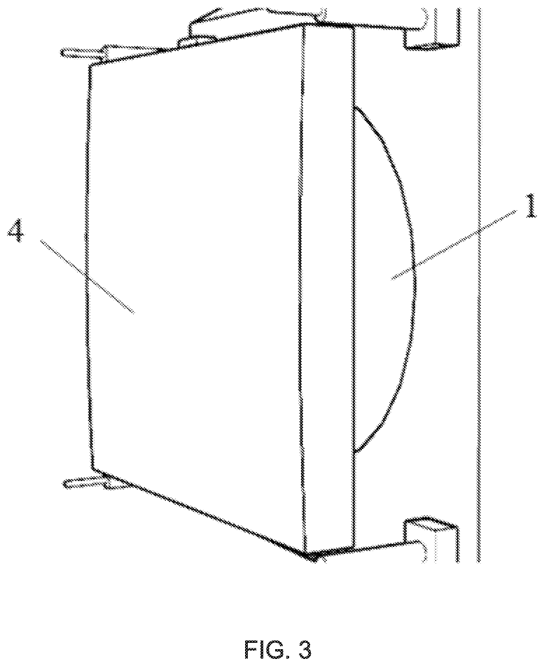

The collection device 8 also includes a load-bearing plate 4, as shown in FIG. 3.

A bottom surface of the load-bearing plate 4 is fixedly connected to the front end surface of the pressure cell 1. When the testing device tests the surrounding rock, a top surface of the load-bearing plate 4 is in contact with the surrounding rock.

The load-bearing plate 4 uniformly transmits a pressure from the surrounding rock to the pressure cell 1, thereby preventing permanent damage or plastic deformation of the pressure cell 1 caused by rough particles on a surface of the surrounding rock, and protecting the pressure cell 1.

The load-bearing plate 4 can be made of Q355 steel, and a bottom surface (a surface in contact with the pressure cell 1) is a flat surface, and a top surface (i.e., a surface in contact with the surrounding rock) is a curved surface. The load-bearing plate 4 has a thickness of about 3 cm.

The bottom surface of the load-bearing plate 4 is square, and a side length of the bottom surface is slightly larger than a diameter of the pressure cell 1. And the side length is sized not to block the displacement meters 2 and thus does not affect the normal operation of the displacement meters 2. The load-bearing plate 4 is fixed on the pressure cell 1, and the curvature of the top surface thereof is the same as that of the surrounding rock of the tunnel, so that the load-bearing plate fits closely with the surrounding rock during the test.

Yield strength of the load-bearing plate 4 should be greater than 300 MPa. A manufactured load-bearing plate 4 should be calibrated in a laboratory first to determine a relationship curve between a bearing load and a deformation of the load-bearing plate, so as to avoid influence on rest results caused by deformation of the load-bearing plate 4.

The collection device 8 further includes a mounting rod 9 and a mounting rod support 5.

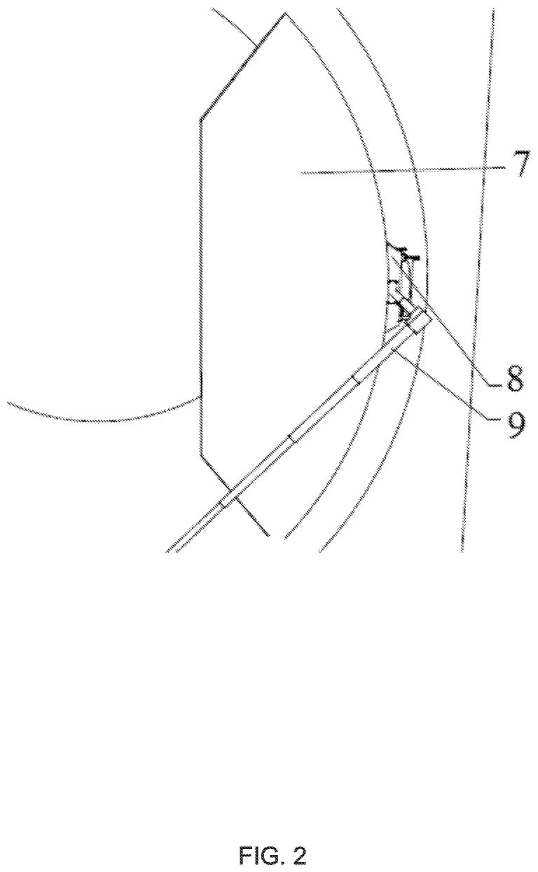

The mounting rod support 5 is arranged on the magnetic base 3. The mounting rod 9 is fixedly connected with the mounting rod support 5 (the mounting rod 9 is not shown in FIG. 1). The collection device 8 is fixedly installed on the outer surface of the gripper 7 through the mounting rod 9, as shown in FIG. 2.

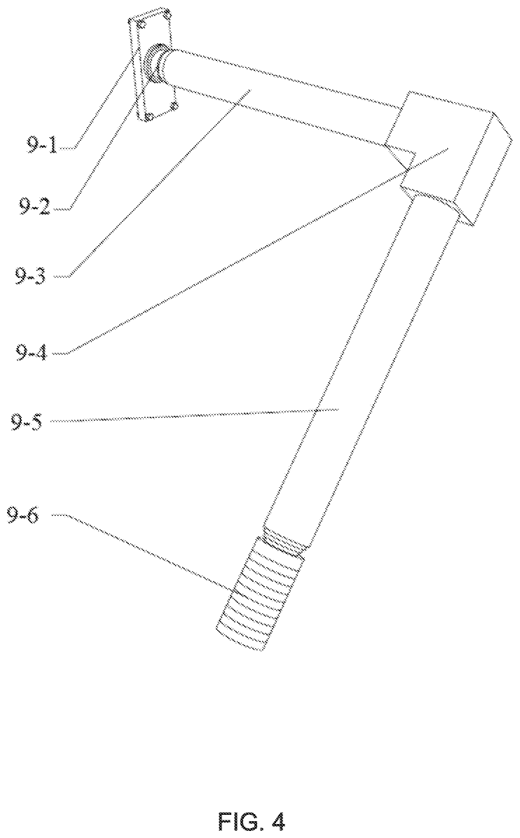

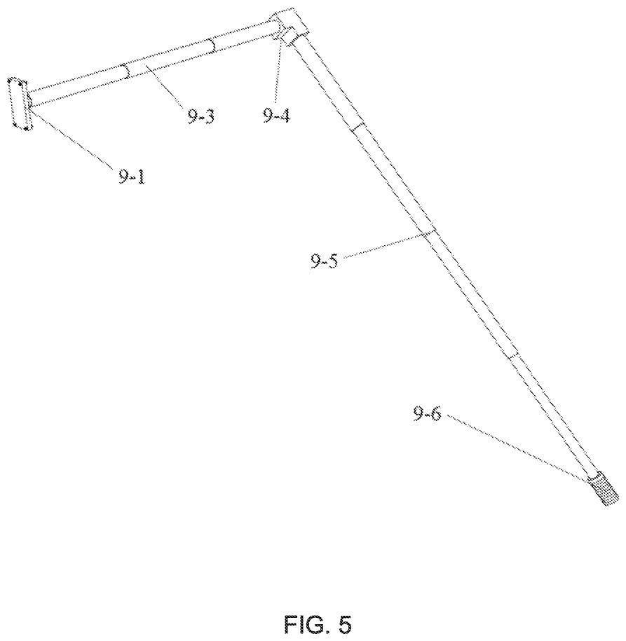

As shown in FIGS. 4-5, the mounting rod 9 includes a telescopic device rod 9-3, a telescopic hand-held rod 9-5, an end connector 9-1, a rotating bearing 9-2, and a corner connector 9-4 and a mounting rod handle 9-6.

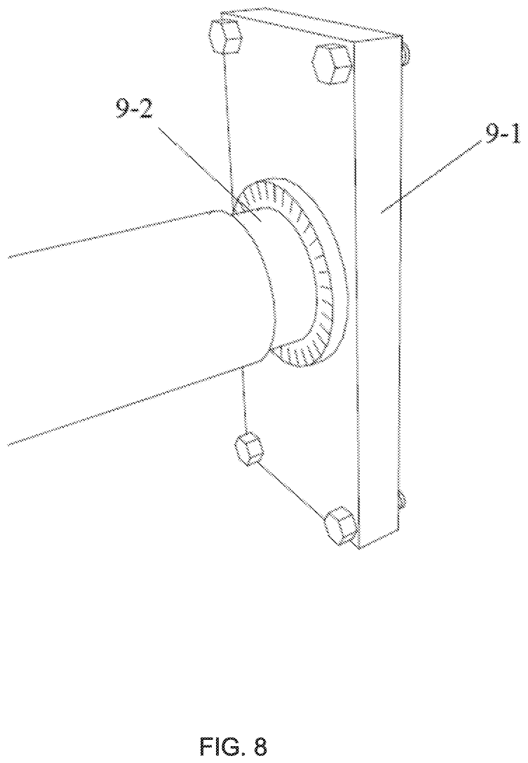

The rotating bearing 9-2 is arranged on the end connector 9-1. The rotating bearing 9-2 enables the end connector to rotate relative to the telescopic device rod, as shown in FIG. 8.

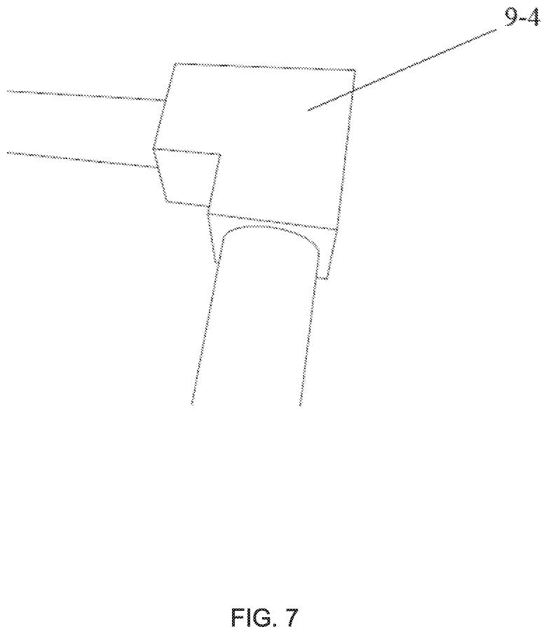

An end of the telescopic device rod 9-3 is connected with an end of the telescopic hand-held rod 9-5 by the corner connector 9-4. The other end of the telescopic device rod 9-3 is connected with the rotating bearing 9-2. The other end of the telescopic hand-held rod 9-5 is connected with the mounting rod handle 9-6. A detailed view of the corner connector 9-4 is shown in FIG. 7.

The end connector 9-1 is fixedly connected with the mounting rod support 5. In some embodiments, the end connector 9-1 is connected with the mounting rod support 5 through a bolt.

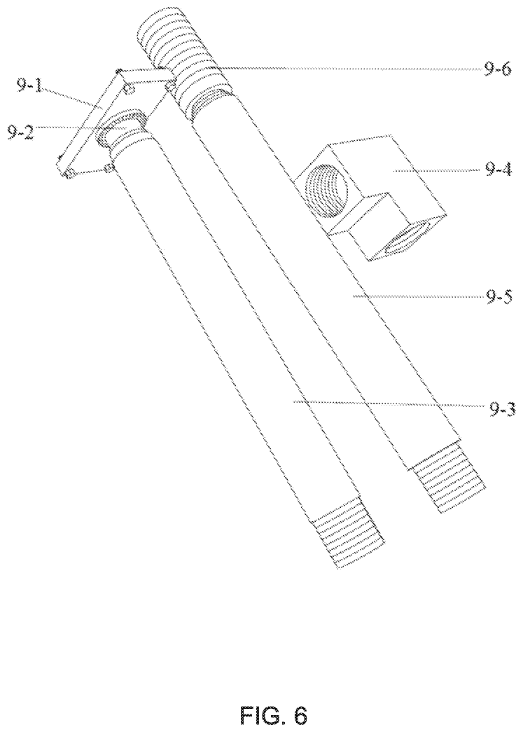

When the collection device 8 is installed, a rotating angle of the rotating bearing 9-2 is adjusted in advance to ensure that the collection device 8 fits closely with the curved surface of the gripper 7 at the time of installing the collection device 8, thereby avoiding an excessive deviation from a predetermined position. After usage of the mounting rod 9, the mounting rod 9 can be disassembled for convenient storage, as shown in FIG. 6.

The control terminal includes a controller, an input device, a memory, a microprocessor, a display and a battery box.

The controller is respectively connected with the pressure cell 1, the multiple displacement meters 2 and the memory. The controller is configured to synchronously collect a pressure measured by the pressure cell 1 and a total compaction displacement measured by the multiple displacement meters 2, and to transmit the pressure and the total compaction displacement collected synchronously to the memory for storage.

The input device is connected with the memory. The input device is configured to obtain a correspondence table of the slope, the elastic modulus and the compressive strength, and configured to store this correspondence table and the mileage information on the surrounding rock in the memory.

The microprocessor is connected to the memory. The microprocessor is configured to: obtain pressures and total compaction displacements at all collection time points from the memory; determine a pressure-displacement curve of the surrounding rock and a slope k of a point corresponding to a maximum pressure on the pressure-displacement curve based on the pressures and the total compaction displacements at all collection time points; obtain the elastic modulus and the compressive strength corresponding to the slope k by matching the correspondence table of the slope, the elastic modulus and the compressive strength; and transmit the pressure-displacement curve, the slope k, the elastic modulus and the compressive strength to the memory for storage.

The microprocessor is also connected with the display. The microprocessor transmits pressures, total compaction displacements, the pressure-displacement curve, the slope k, the elastic modulus and the compressive strength at all collection time points to the display for displaying. In some embodiments, the microprocessor includes integrated circuits. An operator can master operation statuses of the testing device in real time through the display. When a difference between reading values of every two of the four displacement meters is more than 2 mm or an indicating value of the pressure cell 1 is too large, it is determined that a bias pressure failure occurs on the testing device, thereby reminding the operator to stop the current test, check the testing device and perform a test again.

A power input end of the controller, a power input end of the memory, a power input end of the microprocessor and a power input end of the display are all connected to an input end of the integrated power.

The battery box is respectively connected with the pressure cell, the displacement meters, the magnetic base and the input end of the integrated power.

The controller includes an integrated chip, a main switch and multiple sub-switches.

The battery box is connected to an input end of the main switch. An output end of the main switch is connected to an input end of each of the multiple sub-switches. Output ends of the multiple sub-switches are connected with the pressure cell 1, the displacement meter 2, the magnetic base 3 and the integrated power in one-to-one correspondence. The battery box supplies power to the magnetic base 3, the displacement meters 2, the pressure cell 1, and internal components of the control terminal. There are four groups of batteries in the control terminal, and a capacity, a voltage, and a shape of a connector required by the four groups of batteries are determined according to requirements of a power unit.

A control end of the main switch and control ends of the multiple sub-switches are all connected with an integrated chip. The integrated chip controls power on and off of each component. And the integrated chip contains a control algorithm, and controls a collection frequency of the pressure cell 1 and the displacement meters 2 according to the control algorithm to ensure that a time when the pressure cell collects data corresponds to a time when the displacement meters collect data. The sub-switches are configured to turn on each corresponding electrical component separately, which have a protective effect on each electrical component, and determine conveniently that a circuit for a certain electrical component is disconnected.

The collection device also includes a collective wire 6.

A wire used for data transmission and power supply of the pressure cell, wires used for data transmission and power supply of the displacement meters and a wire of the magnetic base are led out together to form the collective wire 6, which is finally connected to the control terminal.

The testing device also includes a storage box.

The storage box is mainly configured for the storage of the testing device, and is made of plastic or other lightweight materials, so as to reduce a portable weight of the testing device. The storage box is a rectangular solid. A cross section of the rectangular solid has a length of about 50 cm, a width of about 30 cm, and a height of about 30 cm. The storage box includes three space areas which are used to store the collection device 8 and the control terminal respectively.

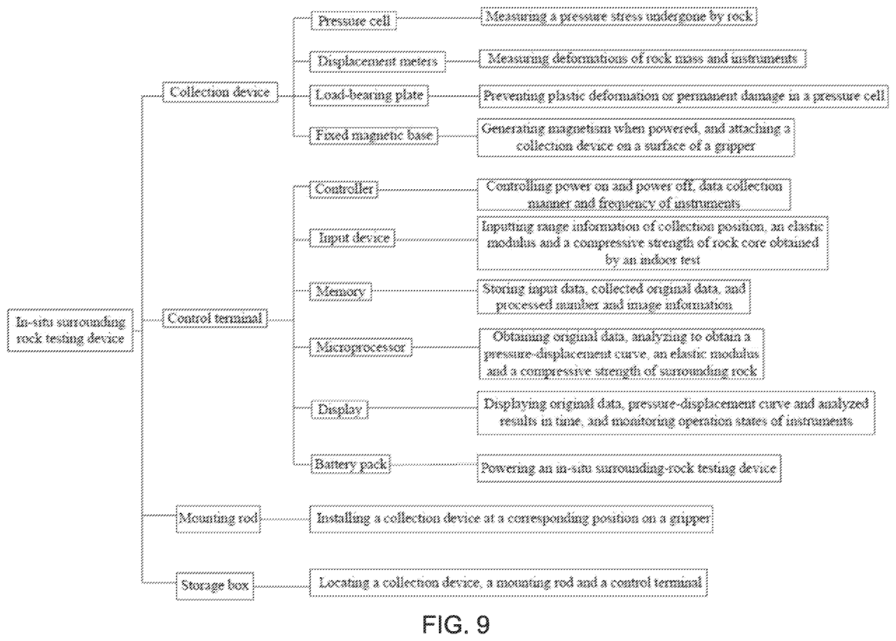

Various structures of the in-situ surrounding rock testing device and functions of the structures are shown in FIG. 9.

The present disclosure provides a testing device for in-situ acquisition of mechanical properties of the surrounding rock for a tunnel boring machine, which is rapid, real-time, accurate and has minimal impact on boring operations. The testing device is easy to operate. It is only necessary to install the testing device on the gripper 7 of the TBM, and a test can be realized through a process in which the gripper 7 presses tightly against the surrounding rock. The test results are real-time and accurate. During the compaction process, a pressure-displacement curve of the surrounding rock can be obtained in real time, and the in-situ test on the surrounding rock effectively ensures accuracy of the test results. The testing device uses the gripper 7 for testing only when the TBM is shut down. The test process has substantially no impact on the normal construction and zero damage to the surrounding rock of an excavated tunnel.

A working process for testing surrounding rock by using the in-situ surrounding rock testing device provided by the present disclosure is as follows:

1. A tester carries the in-situ testing device in place when a pipe segment is being installed on a TBM or a TBM is shut down, ensuring that the gripper 7 is in a retracted state.

2. A location of the surrounding rock to be measured is selected and marked, and a mileage coordinate thereof is recorded.

3. The storage box is opened so as to assemble the mounting rod 9, and the mounting rod 9 is connected with the magnetic base 3.

4. The main switch is turned on so as to check whether the testing device and the display are normal.

5. One end of the mounting rod 9 is held by a hand to attach the collection device 8 to a corresponding position of the gripper 7.

6. The hydraulic device of the gripper 7 is actuated so as to press the gripper 7 against the surrounding rock.

7. Whether a data collection process on the display is abnormal is observed; if it is abnormal, the gripper 7 is retracted, the power supply is turned off to adjust the collection device 8, and the method returns to step 4 for measuring again.

8. Results are recorded after data is collected normally.

9. The gripper 7 is retracted, the mounting rod 9 is held by a hand, and the power supply is turned off, so as to pack up the testing device.

The in-situ surrounding rock testing device provided by the present disclosure has the following technical effects.

(1) The present disclosure provides a device for in-situ testing a relationship between a pressure and a deformation of the surrounding rock of the tunnel at the TBM working site.

(2) The device of the present disclosure is installed on the gripper 7 of the TBM. When the TBM is shut down, through a process in which the gripper 7 presses tightly against the surrounding rock of the tunnel, a test can be completed rapidly. The testing device can be disassembled after the test, and the process of the test does not affect a normal working of the TBM.

(3) The telescopic mounting rod 9 is designed so that the testing device can be installed on the gripper 7 of different sizes. And it is easy to operate the mounting rod 9, and convenient to store and transport the mounting rod 9.

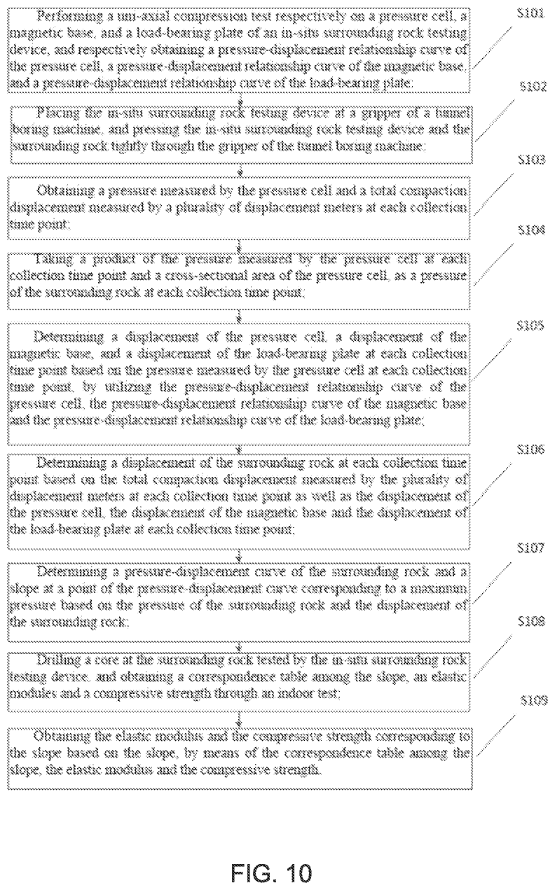

The present disclosure also provides an in-situ surrounding rock testing method. As shown in FIG. 10, the testing method includes the following steps:

In step S101, a uni-axial compression testis performed respectively on a pressure cell 1, a magnetic base 3 and the load-bearing plate 4 of the in-situ surrounding rock testing device, so as to obtain a pressure-displacement relationship curve of the pressure cell 1, a pressure-displacement relationship curve of the magnetic base 3 and a pressure-displacement relationship curve of the load-bearing plate 4.

In step S102, the in-situ surrounding rock testing device is placed at the gripper of the tunnel boring machine, and the in-situ surrounding rock testing device is pressed tightly against the surrounding rock through the gripper of the tunnel boring machine.

A pressure is provided for the in-situ testing device through the gripper, so as to load the surrounding rock slowly. And a compressive stress of the surrounding rock is measured through the pressure cell, and the total compaction displacement is measured through the displacement meters.

In step S103, a pressure measured by the pressure cell 1 and a total compaction displacement measured by the multiple displacement meters 2 at each collection time point are obtained.

In step S104, a product of the pressure measured by the pressure cell at each collection time point and a cross-sectional area of the pressure cell is taken as a pressure of the surrounding rock at each collection time point.

In step S105, based on the pressure measured by the pressure cell 1 at each collection time point, a displacement of the pressure cell 1, a displacement of the magnetic base 3, and a displacement of the load-bearing plate 4 at each collection time point are respectively determined by utilizing the pressure-displacement relationship curve of the pressure cell 1, the pressure-displacement relationship curve of the magnetic base 3, and the pressure-displacement relationship curve of the load-bearing plate 4.

In step S106, based on the total compaction displacement measured by the multiple displacement meters 2 at each collection time point, and the displacement of the pressure cell 1, the displacement of the magnetic base 3 and the displacement of the load-bearing plate 4 at each collection time point, a displacement of the surrounding rock at each collection time point may be determined.

In step S107, a pressure-displacement curve of the surrounding rock and a slope of a point corresponding to a maximum pressure on the pressure-displacement curve are determined based on the pressure of the surrounding rock and the displacement of the surrounding rock.

In step S108, a core is taken at a portion of the surrounding rock that is tested by the in-situ surrounding rock testing device, and a correspondence table of a slope, an elastic modulus and a compressive strength are obtained through an indoor test.

In step S109, based on the slope, the elastic modulus and the compressive strength corresponding to the slope are obtained by the correspondence table of the slope, the elastic modulus and the compressive strength.

Regarding S106, it can include: based on the total compaction displacement measured by the multiple displacement meters 2 at each collection time point, the displacement of the pressure cell 1, the displacement of the magnetic base 3 and the displacement of the load-bearing plate 4 at each collection time point, as well as an average displacement of the multiple displacement meters when an indicating value of the pressure cell 1 starts to be not zero during a test, a displacement of the surrounding rock at each collection time point is determined through the following equation:

$$X = \frac{{\sum }_{i = 1}^{n} {X}_{0 i}}{n} - {X}_{1} - {X}_{2} - {X}_{3} - {X}_{4} \qquad ( 1 )$$

Where X is a displacement of surrounding rock at each collection time point; X₀ᵢ is a total compaction displacement measured by the i^{th} displacement meter at each collection time point; n is the number of displacement meters; X₁ is a displacement of the pressure cell at each collection time point; X₂ is a displacement of the magnetic base at each collection time point; X₃ is a displacement of the load-bearing plate at each collection time point; and X₄ is an average displacement of the multiple displacement meters when an indicating value of the pressure cell starts to be not zero during the test.

In step S107, an equation for calculating the slope of the point corresponding to the maximum pressure on the pressure-displacement curve is:

$$k = \frac{F}{X} \qquad ( 2 )$$

Where k is a slope at a point corresponding to a maximum pressure on the pressure-displacement curve; F is a maximum pressure on the pressure-displacement curve; and X is a displacement of the surrounding rock corresponding to the maximum pressure on the pressure-displacement curve.

During the test, when X reaches Xm or F reaches 100 MPa, the test is shut down, avoiding damage to the surrounding rock, where Xm is a maximum displacement that does not influence a bearing capacity of the surrounding rock, which is determined according to conditions on site.

The in-situ surrounding rock testing method provided by the present disclosure has the following technical effects.

(1) A process of a test is convenient and rapid, and has low cost of manpower and financial resources. An in-situ test can be performed rapidly for the surrounding rock to obtain a pressure-displacement relationship of the surrounding rock during construction of a tunnel.

(2) Compared with a traditional in-situ testing method, the testing method provided by the present disclosure has a faster speed and a lower cost, and measurement results are more timely and accurate.

(3) During a process of tunnel construction, the testing method provided by the present disclosure can help TBM operators to know about the nature of the surrounding rock of the tunnel in a timely manner, reasonably adjust construction parameters and plans, and increase construction safety.

(4) During a measurement process, substantially no additional procedures are added, and a normal construction of the tunnel will not be affected.

Term Explanation:

The term “tunnel” refers to an engineering building buried in the ground, and a usage form of the underground space by human.

The term “TBM” is also referred to as a tunnel boring machine, and is a large and efficient tunnel construction machine that integrates multiple functions such as tunneling, debris removing, guiding, supporting, ventilating and dust removing. The in-situ surrounding rock testing device provided by the present disclosure can be used on a TBM with a gripper, that is, an open-type and double-shield TBM.

The term “surrounding rock” refers to a surrounding rock that undergoes a stress state change due to excavation impact during boring.

The term “gripper”, refers to a component that makes a thrust of advancing a cylinder evenly act on a rock wall, and a TBM is pushed forward by means of a friction force between the gripper and the rock wall.

The term “in-situ test” refers to a test on properties of rock and soil at an original location where the rock and soil is naturally located or basically in an in-situ state and stress condition.

The term “manhole” refers to an opening hole structure through which an operator can enter or exit equipment for installation, maintenance and safety inspection. A TBM manhole is located on a cutter head, and a testing device can enter a tunnel face through the manhole, thereby facilitating the changing and maintenance of a cutter.

The term “tunnel face” is a working face of an excavated tunnel (in coal mining, mining or tunnel engineering) that keeps moving forward.

The term “SPCC cold-rolled sheet” refers to cold-rolled carbon steel sheet and steel strip for general use.

The term “soft magnetic material” refers to a material with magnetization occurring at HC (coercivity) not greater than 1000 A/m. For typical soft magnetic materials, a maximum magnetization can be achieved with a minimum external magnetic field.

The term “plastic deformation” refers to a deformation that cannot be self-recovered. A permanent deformation will occur when a load goes beyond an elastic deformation range; that is, an irreversible deformation or a residual deformation will occur after the load is removed, which is the plastic deformation.

The term “Q355 steel” refers to a low-alloy high-strength structural steel, where “Q” is yield strength; and “355” represents that the yield strength of this steel is 355 MPa.

The term “yield strength” refers to yield limit of a metal material when a yield phenomenon occurs; that is, a stress that resists a small amount of plastic deformation.

The various embodiments in this specification are described in a progressive manner. Each embodiment discusses differences from other embodiments, and the same or similar parts between the various embodiments can be referred to each other.

Specific examples are used to illustrate the principles and implementation of the present disclosure. The description of the above embodiments is only used to help understand the method and core idea of the present disclosure. Furthermore, for those of ordinary skill in the art, according to a concept of the present disclosure, there will be some changes in the specific implementation and scope of application. In summary, the content of this specification should not be construed as limiting the present disclosure.

What is claimed is:

1. An in-situ surrounding rock testing device, comprising a collection device and a control terminal, wherein,
the collection device comprises a pressure cell, a plurality of displacement meters and a magnetic base;
a rear end surface of the pressure cell and the plurality of displacement meters are fixed on a front end surface of the magnetic base;
when the testing device tests a surrounding rock, an outer surface of a gripper of a tunnel boring machine is coupled to a rear end surface of the magnetic base; a front end surface of the pressure cell and the plurality of displacement meters are in contact with the surrounding rock; the pressure cell is configured to measure
pressures to which the surrounding rock is subjected; and the plurality of displacement meters are configured to measure total compaction displacements of the surrounding rock relative to the collection device;
the pressure cell and the plurality of displacement meters are connected to the control terminal; the control terminal is configured to: synchronously collect the pressures measured by the pressure cell and the total compaction displacements measured by the plurality of displacement meters; based on the pressures and the total compaction displacements, determine a pressure-displacement curve of the surrounding rock and a slope of a point on the pressure-displacement curve corresponding to a maximum pressure; and obtain a compressive strength of the surrounding rock based on the slope.

2. The in-situ surrounding rock testing device according to claim 1, wherein the collection device further comprises a load-bearing plate;
a bottom surface of the load-bearing plate is fixedly connected with the front end surface of the pressure cell;
when the testing device tests the surrounding rock, a top surface of the load-bearing plate is in contact with the surrounding rock.

3. The in-situ surrounding rock testing device according to claim 1, wherein the collection device further comprises a mounting rod and a mounting rod support;
the mounting rod support is arranged on the magnetic base;
the mounting rod is fixedly connected with the mounting rod support, and the collection device is fixedly installed on the outer surface of the gripper through the mounting rod.

4. The in-situ surrounding rock testing device according to claim 3, wherein the mounting rod comprises a telescopic device rod, a telescopic hand-held rod, an end connector, a rotating bearing, a corner connector, and a mounting rod handle;
the rotating bearing is arranged on the end connector, the rotating bearing is perpendicularly rotated by 180°,
an end of the telescopic device rod is connected with an end of the telescopic hand-held rod through the corner connector; another end of the telescopic device rod is connected with the rotating bearing; another end of the telescopic hand-held rod is connected with the mounting rod handle,
the end connector is fixedly connected with the mounting rod support.

5. The in-situ surrounding rock testing device according to claim 1, wherein the control terminal comprises a controller, an input device, a memory, a microprocessor, a display, and a battery box;
the controller is respectively connected with the pressure cell, the plurality of displacement meters and the memory; the controller is configured to synchronously collect the pressures measured by the pressure cell and the total compaction displacements measured by the plurality of displacement meters, and configured to transmit the pressures and the total compaction displacements synchronously collected to the memory for storage;
the input device is connected with the memory; the input device is configured to obtain a correspondence table among the slope, an elastic modulus and the compressive strength, and to obtain range information of the surrounding rock; and configured to store the correspondence table among the slope, the elastic modulus and the compressive strength, as well as the range information of the surrounding rock in the memory;
the microprocessor is connected to the memory; the microprocessor is configured to obtain the pressures and total compaction displacements at all collection time points from the memory, and configured to determine the pressure-displacement curve of the surrounding rock based on the pressures and the total compaction displacements at all collection time points and to determine the slope of the point on the pressure-displacement curve corresponding to a maximum pressure; the microprocessor is also configured to obtain an elastic modulus and a compressive strength corresponding to the slope by referring the correspondence table among the slope, the elastic modulus and the compressive strength, and configured to transmit the pressure-displacement curve, the slope, the elastic modulus, and the compressive strength to the memory for storage;
the microprocessor is also connected to the display; the microprocessor is further configured to transmit the pressures and the total compaction displacements at all collection time points, the pressure-displacement curve, the slope, the elastic modulus, and the compressive strength to the display for displaying;
a power input end of the controller, a power input end of the memory, a power input end of the microprocessor, and a power input end of the display are connected to an input end of an integrated power; and
the battery box is respectively connected with the pressure cell, the plurality of displacement meters, the magnetic base and the input end of the integrated power.

6. The in-situ surrounding rock testing device according to claim 5, wherein the controller comprises an integrated chip, a main switch and a plurality of sub-switches;
the battery box is connected to an input end of the main switch; an output end of the main switch is connected to an input end of each of the plurality of sub-switches; output ends of the plurality of sub-switches are connected with the pressure cell, the plurality of displacement meters, the magnetic base and the input end of the integrated power in one-to-one correspondence;
a control end of the main switch and a control end of each of the plurality of sub-switches are connected to the integrated chip.

7. An in-situ surrounding rock testing method, comprising:
performing a uni-axial compression test respectively on a pressure cell, a magnetic base, and a load-bearing plate of an in-situ surrounding rock testing device, and respectively obtaining a pressure-displacement relationship curve of the pressure cell, a pressure-displacement relationship curve of the magnetic base, and a pressure-displacement relationship curve of the load-bearing plate;
placing the in-situ surrounding rock testing device at a gripper of a tunnel boring machine, and pressing the in-situ surrounding rock testing device and the surrounding rock tightly through the gripper of the tunnel boring machine;
obtaining a pressure measured by the pressure cell and a total compaction displacement measured by a plurality of displacement meters at each collection time point;
taking a product of the pressure measured by the pressure cell at each collection time point and a cross-sectional area of the pressure cell, as a pressure of the surrounding rock at each collection time point;
determining a displacement of the pressure cell, a displacement of the magnetic base, and a displacement of the load-bearing plate at each collection time point based on the pressure measured by the pressure cell at each collection time point, by utilizing the pressure-displacement relationship curve of the pressure cell, the pressure-displacement relationship curve of the magnetic base and the pressure-displacement relationship curve of the load-bearing plate;
determining a displacement of the surrounding rock at each collection time point based on the total compaction displacement measured by the plurality of displacement meters at each collection time point as well as the displacement of the pressure cell, the displacement of the magnetic base and the displacement of the load-bearing plate at each collection time point;
determining a pressure-displacement curve of the surrounding rock and a slope at a point of the pressure-displacement curve corresponding to a maximum pressure based on the pressure of the surrounding rock and the displacement of the surrounding rock;
drilling a core at the surrounding rock tested by the in-situ surrounding rock testing device,
obtaining a correspondence table among the slope, an elastic modulus and a compressive strength through an indoor test;
obtaining the elastic modulus and the compressive strength corresponding to the slope based on the slope, by means of the correspondence table among the slope, the elastic modulus and the compressive strength.

8. The in-situ surrounding rock testing method according to claim 7, wherein the determining a pressure-displacement curve comprises determining a partial pressure-displacement curve of the surrounding rock and a slope at a point of the partial pressure-displacement curve corresponding to a maximum pressure based on the pressure of the surrounding rock and the displacement of the surrounding rock;
wherein the drilling comprises drilling a core at the surrounding rock tested by the in-situ surrounding rock testing device, to obtain an elastic modulus and a compressive strength of the surrounding rock by performing an indoor laboratory test on the core, and in turn obtaining a global pressure-displacement curve and a slope of the global pressure-displacement curve of the surrounding rock based on the partial pressure-displacement curve;
wherein the obtaining a correspondence table comprises obtaining a correspondence table among the elastic modulus, the compressive strength and the slope of the global pressure-displacement curve of the surrounding rock by repeating the drilling a core;
wherein the obtaining the elastic modulus comprises obtaining an elastic modulus and a compressive strength of a surrounding rock at a new location to be detected, based on the slope and the global pressure-displacement curve by means of the correspondence table.

9. The in-situ surrounding rock testing method according to claim 7, wherein determining a displacement of the surrounding rock at each collection time point, based on the total compaction displacement measured by the plurality of displacement meters at each collection time point, as well as the displacement of the pressure cell, the displacement of the magnetic base and the displacement of the load-bearing plate at each collection time point, comprises:
determining a displacement of the surrounding rock at each collection time point, based on the total compaction displacement measured by the plurality of displacement meters at each collection time point, the displacement of the pressure cell, the displacement of the magnetic base and the displacement of the load-bearing plate at each collection time point, through following equation:
$$X = \frac{{\sum }_{i = 1}^{n} {X}_{0 i}}{n} - {X}_{1} - {X}_{2} - {X}_{3} - {X}_{4} ; \qquad ( 1 )$$
wherein X is the displacement of the surrounding rock at each collection time point; X₀ᵢ is the total compaction displacement measured by the i^{th} displacement meter at each collection time point, n is a number of displacement meters; X₁ is a displacement of the pressure cell at each collection time point; X₂ is a displacement of the magnetic base at each acquisition time point; X₃ is a displacement of the load-bearing plate at each collection time point; and X₄ is an average displacement of the plurality of displacement meters when an indicating value of the pressure cell is not zero during the test.

10. The in-situ surrounding rock testing method according to claim 7, wherein after the determining a displacement of the surrounding rock at each collection time point based on the total compaction displacement measured by the plurality of displacement meters at each collection time point, as well as the displacement of the pressure cell, the displacement of the magnetic base and the displacement of the load-bearing plate at each collection time point, the testing method further comprises:
when the displacement of the surrounding rock is equal to a maximum displacement threshold or a pressure of the surrounding rock is equal to 100 MPa, the test is stopped.

11. The in-situ surrounding rock testing method according to claim 7, wherein an equation for calculating the slope at the point on the pressure-displacement curve corresponding to the maximum pressure is:
$$k = \frac{F}{X} ; \qquad ( 2 )$$
wherein k is the slope of the point on the pressure-displacement curve corresponding to the maximum pressure; F is the maximum pressure on the pressure-displacement curve; and X is the displacement of the surrounding rock corresponding to the maximum pressure on the pressure-displacement curve.
